(19) United States
(12) Patent Application Publication
(10) Pub. No.: US 2014/0294666 A1
(43) Pub. Date: Oct. 2, 2014

(54) Air-Flow Activated Germicidal UV-C Lights in HVAC System with Electromagnetic Induction and Other Proximity Sensor Technologies

(71) Applicant: Benjamin D. Liu, Carmel, IN (US)

(72) Inventor: Benjamin D. Liu, Carmel, IN (US)

(21) Appl. No.: 13/853,566

(22) Filed: Mar. 29, 2013

(51) Int. Cl. A61L 9/20

(52) U.S. Cl. CPC A61L 9/20
USPC 422/4; 250/432 R

(57) ABSTRACT

UV-C light assembly is designed to kill germs (bacteria, molds, protozoa, virus, and yeast) in the forced airstreams of HVAC systems. Thus the UV-C light kills and prevents the spreading of germs into other rooms or spaces. An air-flow activated switch is invented for turning on the UV-C lights when airstream passes through and turning off when airstream stops the flowing in the HVAC systems. The invented air-flow activated switches include electromagnetic induction and other proximity sensor switches such as infrared, ultrasonic, capacitive, laser, radar, inductive and etc. The UV-C light assembly is installed inside air duct. The UV-C germicidal assembly is an easy add-on to an existing HVAC system for indoor air purification. The UV-C light sources are either LEDs or discharge lamps.

### BACKGROUND OF THE INVENTION

It is well known that sunlight purifies air. More specifically, it is the light in the UV-C wavelength range (around 254 nm) that purifies air. UV-C light is germicidal by deactivating the DNAs of viruses, bacteria, and dust mites. However, this natural germicidal process doesn't occur indoors. Many UV-C germicidal systems are on the market for indoor uses such as UV-C sanitizing wands, UV-C housings used inside HVAC systems.

### SUMMARY OF THE INVENTION

This invention designed an UV-C light assembly for air purification inside HVAC systems with two types of air-flow activated switches: electromagnetic induction and proximity sensor activated switches.

We use light-emitting diodes (LEDs) or discharge (fluorescent) light tubes in the wavelength range of UV-C centered at about 254 nm. The UV-C wavelength is peaked at 254 nm. The light assembly is placed inside air duct of a HVAC system. When the HVAC system starts to work, the HVAC fan drives the air in the duct to flow. We invented two categories of air-stream activated switches for the UVC light assembly: (1) electromagnetic induction switch, and (2) proximity sensor switches such as infrared, ultrasonic, laser, radar, capacitive, inductive and etc. These switches are activated by the air flows inside HVAC ducts, i.e., the air-flow turns on the switch of the UV-C light. The UV-C light purifies the air that flow through the light. When the HVAC fan stops, the switches turn off the UV-C light assembly.

The UV-C light assembly is designed in such a way that it can be installed easily inside HVAC systems.

This UV-C light assembly is suitable for any central HVAC systems.

### BRIEF DESCRIPTION OF THE DRAWINGS OF THE INVENTION

FIG. 1 is a structure of electromagnetic induction activated UV-C light assembly.

FIG. 2 is a structure of proximity sensor activated UV-C light assembly.

FIG. 3 is an illustration of a typical UV-C light assembly circuit.

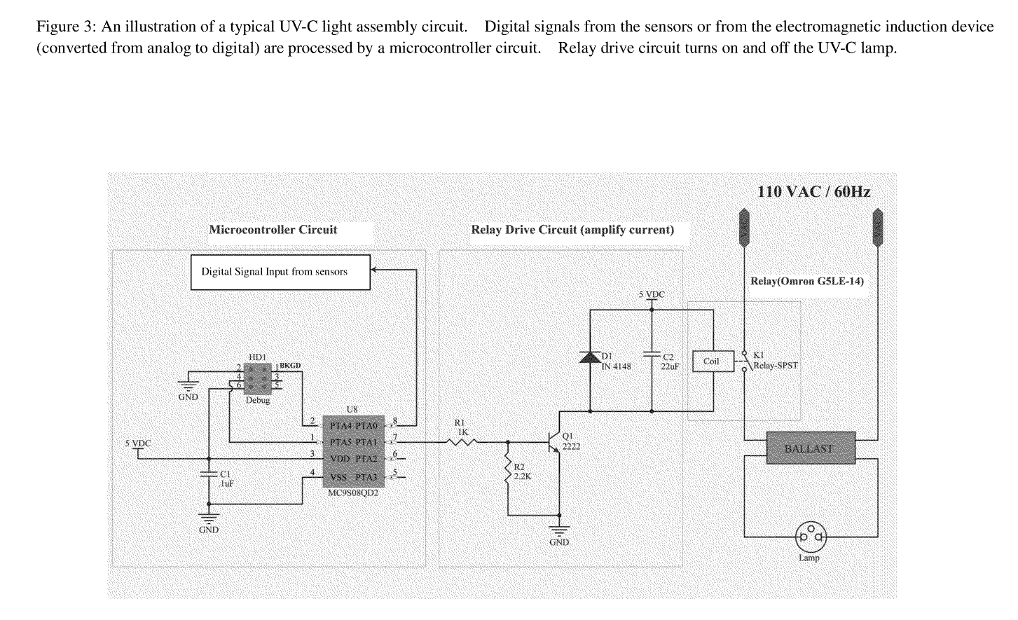

### DETAILED DESCRIPTION OF THE INVENTION

### 1. The Physical Structure

The invented UV-C light assembly consists of units as follows:

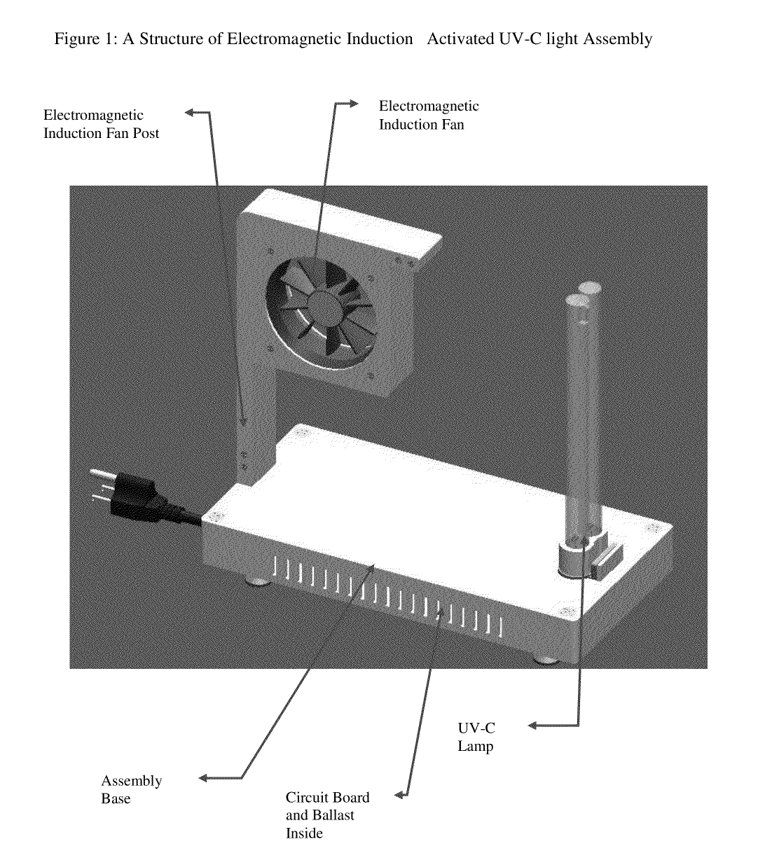

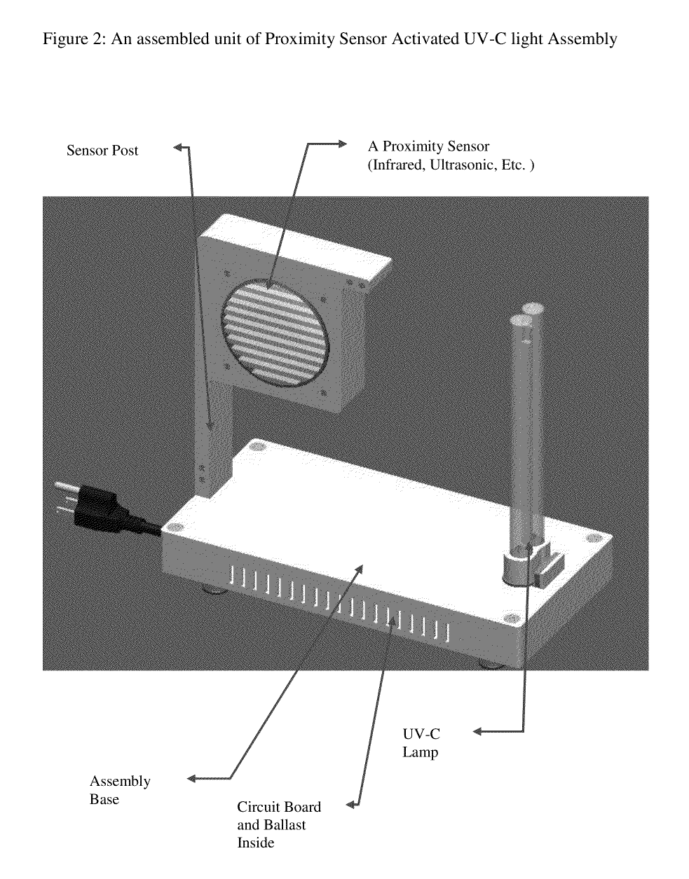

### (a) Assembly Base (FIGS. 1 and 2)

A box that can be made of metal or plastics holds all other parts together to form the assembly.

### (b) Circuit Board and Lamp Base (FIGS. 1 and 2)

The circuit is etched on the board with necessary electronic components and lamp base (of discharge lamp or LEDs) welded.

### (c) Electromagnetic Induction Fan (FIG. 1)

A fan with magnets and conductor wire coil is placed in the air stream. When air flows in the duct, it forces the induction fan to rotate. Thus, electrical current is induced to flow through the coil wire. We design an electronic circuit to turn on the power for the light assembly by the induced electrical current.

### (d) Proximity Sensor Switches (FIG. 2)

Instead of using electromagnetic induction fan as a sensor of the air flow inside ducts, we use other types of proximity sensor switches. The proximity switches include infrared sensor, ultrasonic, laser, radar, capacitive, inductive, etc.

(e) UV-C discharge lamp (FIGS. 1 and 2)

### 2. Operation of Air-Flow Activated Germicidal UV-C Light Assembly

Air-flow activated germicidal UV-C light assembly (light assembly) is designed to be used with central HVAC systems. The UV-C light assembly is installed inside air ducts. When air flows, the light assembly turns on. When air flow stops, the light assembly turns off. When the light assembly turns on, it emits UV-C light and illuminates a section of the duct. Air flowing through this light illuminated section is purified by UV-C light. Here is how this light assembly works:

(a) UV-C lamps (or LEDS) are installed on the circuit board according to FIGS. 1 and 2). The light assembly is either totally installed inside or only the lamp and the senor installed inside the HVAC duct.

(b) When the air in the duct doesn't flow, meaning the HVAC system is off, the induction fan or other air-flow-induced proximity action devices stay in off positions. Thus, the UV-C assembly is not turned on.

(c) When the air in the duct flows, meaning the HVAC system is on, the induction fan is pushed to spin. The magnetically induced current in the fan conductor wire (coil) sends an electric current to an analog to digital converter. Then, the digital converter sends “on” signal to the microcontroller circuit. The microcontroller turns on the relay drive circuit, and thus the lamp switch. When the switch is on, the lamp emits UV-C light (FIG. 1).
The same switching-on action occurs for other proximity sensor switches. When the air in the duct flows, the air-flow-induced devices begin to operate. These operations change the environments of the proximity sensors. The sensors send “on” signal to the microcontroller, and thus turn on the UV-C lamps (FIG. 2).

(d) While the light assembly is on, the air flows through the illuminated section. The UV-C light purifies the airstream.

### CROSS-REFERENCES FOR THE INVENTION OF AIR-FLOW ACTIVATED GERMICIDAL UV-C LIGHT ASSEMBLY

U.S. Pat. No. 7,976,195 Engel, et al

An UVC lamp and reflector/shield assembly is designed to be used in a commercial HVAC unit. The reflector in the assembly creates an illuminating pattern. The outer surface of the assembly provides shielding for the UVC lamp from the air flow.

U.S. Pat. No. 8,004,166 Boehme

The author designed a mercury based UVC lamp that prevents the formation of a cold spot in the lamp.

U.S. Pat. Nos. 8,252,099 and 8,252,100 Worrilow

An air purifier is designed that includes a housing with an inlet for receiving air and an outlet for exhausting air. The housing has a particle pre-filtration, UV filtration, and volatile organic compound post filtration. This purifier is intended to use in research laboratories.

U.S. Pat. No. 7,837,933 Sevack, et al

This patent is similar to U.S. Pat. No. 7,976,195

U.S. Pat. No. 7,740,686 Metteer

A modular ductwork assembly decontaminates an air stream circulating with a HVAC system. The assembly includes an ionizing module, sterilization module, ozone treatment module. The sterilization module uses UVC light.

U.S. Pat. No. 7,278,272 Huston, et al

A germicidal UVC lamp is mounted near evaporator coil in a HVAC system to sterilize the air and surfaces of the coil.

U.S. Pat. No. 7,175,814 Dionisio

A cartridge device containing UVC for air disinfection comprises individual UV bulb, filter, ballast and electrical components.

1. This author invented an air-flow activated germicidal UV-C light in HVAC system with electromagnetic induction and other proximity sensor technologies.

2. The author uses the electric current generated through electromagnetic induction from the conductor coils and magnets in a fan placed in the air stream of a HVAC duct as input signals for the microcontroller and relay drive to turn on or to turn off the UV-C light.

3. In the same token, the author uses infrared sensor (a type of proximity sensor) and an air-flow-induced action device, for example a shutter, to provide input signals for the microcontroller and relay drive to turn on or to turn off the UV-C light.

4. In the same token, the author uses ultrasound sensor (another type of proximity sensor) and an air-flow-induced action device, for example a spring loaded movable object, to provide input signals for the microcontroller and relay drive to turn on or to turn off the UV-C light.

5. According to claims 2, 3, 4, the author can use other types of proximity sensors such as laser, radar, capacitive, inductive, sonar, and etc. as air-flow-induced action devices to provide input signals for the microcontroller and relay drive to turn on or to turn off the UV-C lights.

6. UV-C light illuminates a section of HVAC duct. The UV-C light purifies airstream that passes through the section by killing the viruses, bacteria, and dust mites.

7. According to claim 2, 3, 4, 5, the electromagnetic induction device or other proximity sensors turn on the UV-C light assembly when the air flows inside the HVAC duct. The electromagnetic induction device or other proximity sensors turn off the UV-C light assembly when the air doesn't flow inside the HVAC duct.

8. According to claim 2, 3, 4, 5, UV-C light lamps and sensors can be placed anywhere inside HVAC ducts where airstream passes.

9. According to claim 2, 3, 4, 5, 8, UV-C assembly can be placed wholly inside the HVAC duct, or only the UV-C lamp and sensor are place inside the UV-C duct while the base and other parts of the assembly are mounted outside of the duct.
